(19) United States
(12) Patent Application Publication
(10) Pub. No.: US 2010/0298756 A1
(43) Pub. Date: Nov. 25, 2010

(54) Tampon Having A Visual Indicator

(76) Inventors: Jeanne Marie Hughes, Loveland, OH (US); Devin William Baldridge, Cincinnati, OH (US); Virginia Anne Lenahan, Cincinnati, OH (US); Cynthia Marie Sims, Liberty Township, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/848,353

(22) Filed: Aug. 2, 2010

Related U.S. Application Data
(63) Continuation of application No. 11/799,891, filed on May 3, 2007.

(51) Int. Cl. A61F 13/20; A61F 13/42; A61F 13/34

(52) U.S. Cl. 604/11; 604/361; 604/385.18

(57) ABSTRACT

A tampon having one or more benefit indicators is provided. The tampon can include a tampon body having an insertion end, a withdrawal end, and an outer surface. The tampon can have one or more visually perceptible benefit indicators that are visible pre-use, wherein the one or more benefit indicators communicate one or more benefits of the tampon to a user. In addition, or alternatively, the tampon can include one or more visually perceptible benefit indicators that are at least partially visible post-use.

### CROSS REFERENCE TO RELATED APPLICATION

This application is a continuation of U.S. patent application Ser. No. 11/799,891, filed May 3, 2007.

### FIELD OF THE INVENTION

This invention relates to tampons, and more particularly to tampons having one or more visual indicators.

### BACKGROUND OF THE INVENTION

Tampons for feminine hygiene are typically used within a woman's vagina to absorb body exudates, such as menstrual fluids. Tampons are generally categorized by absorbency level to enable women to select a tampon that provides optimal leakage protection, that is, the tampon should prevent menstrual discharges from leaking out of the user's body. Absorbency level is generally regulated and publicized, such as, e.g., on the tampon package, wrapper, and/or applicator of the tampon, to assist women in selecting the proper tampon. Women often experience unexpected leakage, however, even though they believe they have chosen a tampon with the correct absorbency level. This unexpected leakage can contribute to a feeling of anxiety in the consumer during use.

Although currently available tampons can contain enhanced features that provide improved leakage protection, the consumer may not be aware of these features when viewing the tampon. Typical tampons are uniform in color such that leakage protection features or other features may not be recognized by a consumer. Even if a feature is visible to a consumer, the consumer may not appreciate the function of that portion of the tampon. As such, even though a tampon can contain enhanced features, if a consumer does not appreciate these features, her anxiety about leakage may not be alleviated.

As such, it would be desirable to provide a tampon that can effectively communicate to the user attributes of the tampon. It would further be desirable to provide a tampon that can effectively communicate to the user attributes such as, e.g., a perception of comfort and/or leakage protection.

### SUMMARY OF THE INVENTION

A tampon having one or more benefit indicators is provided. The tampon can comprise a tampon body having an insertion end, a withdrawal end, and an outer surface. The tampon can include one or more visually perceptible benefit indicators that are visible pre-use, wherein the one or more benefit indicators communicate one or more benefits of the tampon to a user. In addition, or alternatively, the tampon can include one or more visually perceptible benefit indicators that are at least partially visible post-use. In certain embodiments, the one or more benefit indicators can include one or more protection indicators.

A tampon comprising a visually perceptible benefit indicator that is visible pre-use and that is disposed on the outer surface of the tampon body substantially between the insertion end and the withdrawal end is also provided. In certain embodiments, the benefit indicator can be a shade of a color. The first benefit indicator can communicate one or more benefits of the tampon to a user.

### BRIEF DESCRIPTION OF THE DRAWINGS

FIGS. 1A and 1B are perspective views of a tampon.

FIGS. 2A and 2B are perspective views of a tampon.

FIGS. 3A and 3B are perspective views of a tampon.

FIGS. 4A and 4B are perspective views of a tampon.

FIGS. 5A and 5B are perspective views of a tampon.

FIGS. 6A and 6B are perspective views of a tampon.

FIGS. 7A and 7B are perspective views of a tampon.

FIGS. 8A and 8B are perspective views of a tampon.

FIG. 9 is a side view of a tampon.

FIG. 10 is a side view of a tampon.

FIG. 11 is a side view of a tampon.

FIG. 12 is a side view of a tampon.

FIG. 13 is a side view of a tampon.

FIG. 14 is an end view of a tampon.

FIG. 15 is an end view of a tampon.

FIG. 16 is an end view of a tampon.

### DETAILED DESCRIPTION OF THE INVENTION

The present invention relates to tampons having one or more benefit indicators. In certain embodiments, the one or more benefit indicators can be one or more protection indicators. Such tampons can communicate to the user benefits of the tampon, such as, e.g., protection features, comfort features, softness features, or other features, and/or can communicate a perception of improved leakage protection, softness, and/or comfort. In certain embodiments, a benefit indicator can communicate to a user tampon features other than size and/or absorbency level. Such benefit indicators can allow a user to become aware of the benefits and features of the tampon while viewing the tampon itself. Benefit indicators, such as, e.g., protection indicators, can further provide a user with confidence in her choice of leakage protection and/or can reduce the anxiety a user may feel regarding leakage. Benefit indicators can be used to communicate the presence of any suitable feature of a tampon, such as, e.g., comfort features, softness, scent, lubrication, an absorbent core, layers, a secondary absorbent, an overwrap, channels, grooves, protrusions, apertures, and/or a skirt. In certain embodiments, benefit indicators can be used to communicate the perception of a feature of a tampon, such as, e.g., a protection feature, such as, e.g., a fluid barrier, paths of fluid flow, wicking, fluid containment features, and/or any other suitable feature, and/or a comfort feature, such as, e.g., ease of insertion. In certain embodiments, the benefit indicator can be visible pre-use to the user. In addition, or alternatively, all or a portion of the benefit indicator can be visible post-use, such as, e.g., after expansion and/or removal of the tampon.

As used herein, the term “tampon” refers to any type of absorbent structure such as, e.g., an absorbent mass, that can be inserted into the vaginal canal or other body cavity, such as, e.g., for the absorption of fluid therefrom, to aid in wound healing, and/or for the delivery of materials, such as moisture or active materials such as medicaments. In general, the term “tampon” is used to refer to a finished tampon after the compression and/or shaping process.

As used herein, the term “pledget” refers to an absorbent material prior to the compression and/or shaping of the material into a tampon. Pledgets are sometimes referred to as tampon blanks or softwinds.

As used herein, the term “absorbency level” refers to the grams of fluid that typically can be absorbed by a tampon. Any suitable classification for absorbency level, such as, e.g., past, current, and future U.S., European, or other classification schemes, can be used. One example of a absorbency level classification scheme includes, e.g., a junior or light absorbency level corresponding to a tampon absorbency less than or equal to 6 g of fluid as measured by the syngyna test, a regular absorbency level generally corresponding to 6 g to 9 g of fluid, a super absorbency level generally corresponding to 9 g to 12 g of fluid, a super plus absorbency level generally corresponding to 12 g to 15 g of fluid, and an ultra absorbency level generally corresponding to 15 g to 18 g of fluid as measured by the syngyna test. Another example of an absorbency level classification scheme includes, e.g., one droplet corresponding to a tampon absorbency level less than or equal to 6 g of fluid as measured by the syngyna test, two droplets corresponding to 6 g to 9 g of fluid, three droplets corresponding to 9 g to 12 g of fluid, four droplets corresponding to 12 g to 15 g of fluid, five droplets corresponding to 15 g to 18 g of fluid, and six droplets corresponding to 18 g to 21 g of fluid as measured by the syngyna test. As used herein, the term “vaginal canal” refers to the internal genitalia of the human female in the pudendal region of the body. The terms “vaginal canal” or “within the vagina” as used herein are intended to refer to the space located between the introitus of the vagina and the cervix.

As used herein, the term “applicator” refers to a device or implement that facilitates the insertion of a feminine hygiene product, such as, e.g., a tampon or pessary, into an external orifice of a mammal Suitable applicators include, e.g., telescoping, tube and plunger, and compact applicators.

As used herein, the term “insertion end” refers to the portion of the tampon or applicator including the end that is intended to enter the vaginal canal first when inserting the tampon or applicator into the vaginal canal.

As used herein, the term “withdrawal end” refers to the portion of the tampon or applicator opposite the insertion end.

As used herein, the term “benefit indicator” refers to a visual indicator that can communicate the presence and/or function of a benefit or feature of a tampon to a user. In certain embodiments, the benefit indicator can communicate a benefit or feature, or the perception of a benefit or feature, of a tampon other than size and/or absorbency level. Alternatively, or in addition, the benefit indicator can be, for example, a comfort indicator, a softness indicator, and/or a protection indicator.

As used herein, the term “protection indicator” refers to a visual indicator that can communicate the presence and/or function of a protection feature in a tampon to a user. In certain embodiments, a protection indicator can refer to a visual indicator that can communicate the perception of a protection feature in a tampon to a user.

As used herein, the term “protection feature” refers to a feature of a tampon other than tampon size or tampon absorbency level. Protection features can include, e.g., an absorbent core, layers, a secondary absorbent, channels, grooves, protrusions, apertures, a skirt, or any other protection features. In certain embodiments, a protection feature can include the perception of a feature, such as, e.g., a fluid barrier, paths of fluid flow, wicking, fluid containment features, and/or any other suitable feature.

As used herein, the term “visual indicator” refers to one or more indications or signals visually perceptible to a user. Visual indicators can be only visually perceptible, i.e., visually perceptible visual indicators. By “visually perceptible” is meant that a human viewer can visually discern the visual indicator with the unaided eye (excepting standard corrective lenses adapted to compensate for near-sightedness, farsightedness, or astigmatism, or other corrected vision) in lighting at least equal to the illumination of a standard 100 watt incandescent white light bulb at a distance of 1 meter. By “only visually perceptible” is meant that the visual indicators cannot be readily perceived by touch. Therefore, channels, grooves, protrusions, and the like are generally not considered to be only visually perceptible. However, one or more visual indicators that are only visually perceptible can be used in conjunction with such channels, grooves, protrusions, and the like. For example, one or more only visually perceptible visual indicators, such as, e.g., one or more colors, can be used in conjunction with one or more grooves.

As used herein, the term “color” includes any color, such as, e.g., white, black, red, orange, yellow, green, blue, violet, brown, and/or any other color.

As used herein, “compression” refers to the process of pressing, squeezing, compacting or otherwise reducing the size, shape, and/or volume of a material to obtain a tampon having a vaginally insertable shape. The term “compressed” refers to the state of a material or materials subsequent to compression.

The “outer surface” of a tampon refers to the visible surface of the compressed and/or shaped tampon prior to use and/or expansion. At least part of the outer surface can be smooth or alternatively can have topographical features, such as ribs, grooves, channels, spiraling ribs, a mesh pattern, or other topographical features. In certain embodiments, the tampon can include an overwrap and the outer surface of the overwrap can be visible to a user. In addition, in certain embodiments, when the tampon has an overwrap, the outer surface of the tampon may or may not be visible to a user through the overwrap.

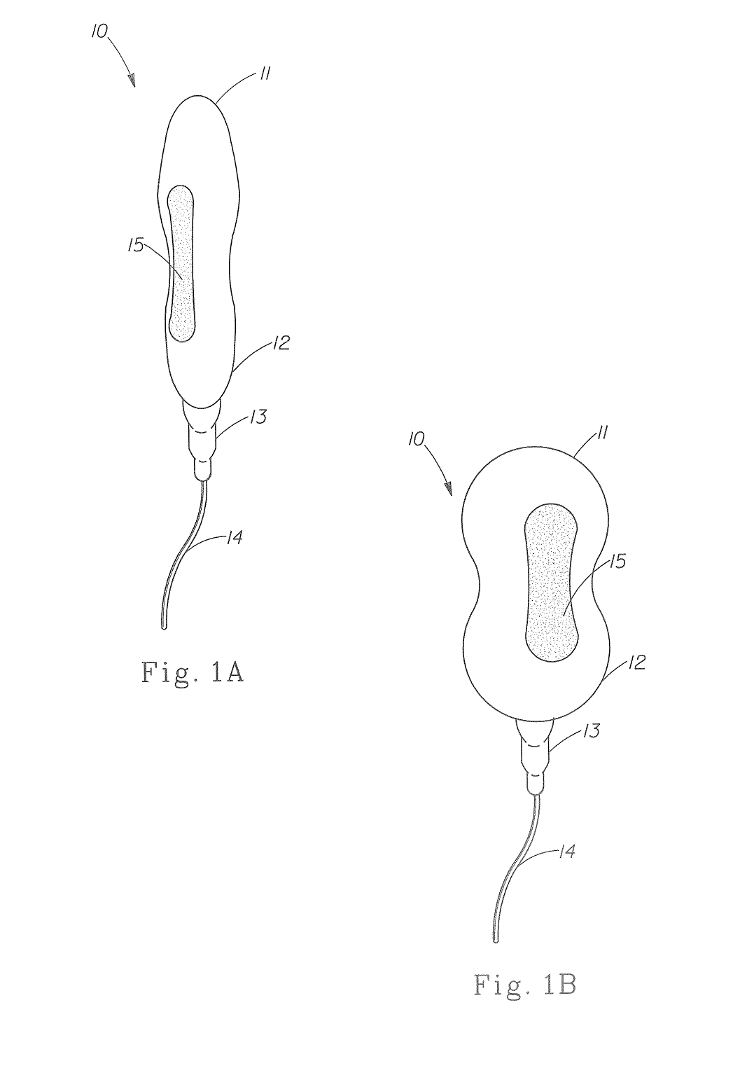

FIG. 1A shows one embodiment of a tampon 10. The tampon 10 has an insertion end 11 and a withdrawal end 12. In this embodiment, the tampon 10 has a secondary absorption member 13. The tampon 10 also has a withdrawal member 14, such as, e.g., a withdrawal cord. FIG. 1A shows the tampon 10 in a compressed state, such as, e.g., prior to use by a user. As shown in FIG. 1A, the tampon 10 has a benefit indicator 15. The benefit indicator 15 is centrally located along the outer surface of the tampon 10. In certain embodiments, the benefit indicator 15 can be a protection indicator and can function, for example, to communicate to a user protection features of the tampon, such as, e.g., the fluid-containment properties of the tampon 10, such as, e.g., that the menstrual fluid can be drawn into the center of the tampon 10. In certain embodiments, the benefit indicator 15 can be a color, such as, e.g., blue and/or green. FIG. 1B shows the tampon 10 of FIG. 1A as expanded, such as, for example, after the tampon 10 is contacted with liquid. As shown in FIG. 1B, the benefit indicator 15 can be substantially maintained post-expansion. In certain embodiments, a benefit indicator 15 having a similar shape and/or location pre-expansion and post-expansion can communicate to a user that the benefit, such as, e.g., the perception of a fluid-containment core, can continue during use of the tampon 10.

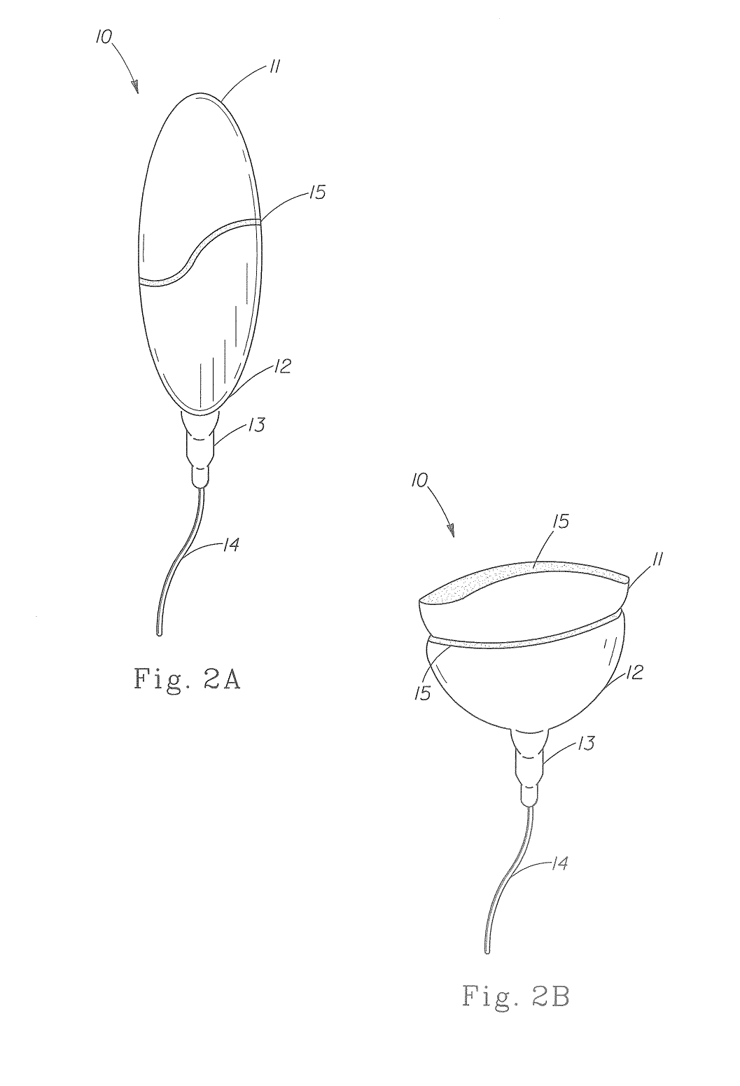

FIG. 2A shows one embodiment of a tampon 10. The tampon 10 has an insertion end 11 and a withdrawal end 12. In this embodiment, the tampon 10 has a secondary absorption member 13. The tampon 10 also has a withdrawal member 14. FIG. 2A shows the tampon 10 in a compressed state. As shown in FIG. 2A, the tampon 10 has a first benefit indicator 15. In this embodiment, the first benefit indicator 15 can be a protection indicator disposed along one or more layers of the tampon 10 such that the first benefit indicator 15 can, for example, communicate to a user protection features of the tampon, such as, e.g., the presence of multiple layers of protection in the tampon 10. FIG. 2B shows the tampon 10 of FIG. 2A as expanded, such as, for example, after the tampon 10 is contacted with liquid. As shown in FIG. 2B, the benefit indicator 15 can have a similar shape and location post-expansion and pre-expansion. As shown in FIG. 2B, in certain embodiments, the tampon 10 can have a second benefit indicator 15 that can be located substantially on the interior of the compressed tampon 10 such that the second benefit indicator 15 may not be substantially visible to a user prior to use of the tampon 10 and can be primarily visible upon expansion of the tampon 10. In certain embodiments, the second benefit indicator 15 can be primarily visible from the insertion end 11 of the tampon 10. A benefit indicator 15, such as, e.g., a protection indicator, that is primarily visible upon expansion of the tampon 10 can communicate to a user protection features, such as, e.g., a perception of fluid movement to and/or containment within the core of the tampon 10.

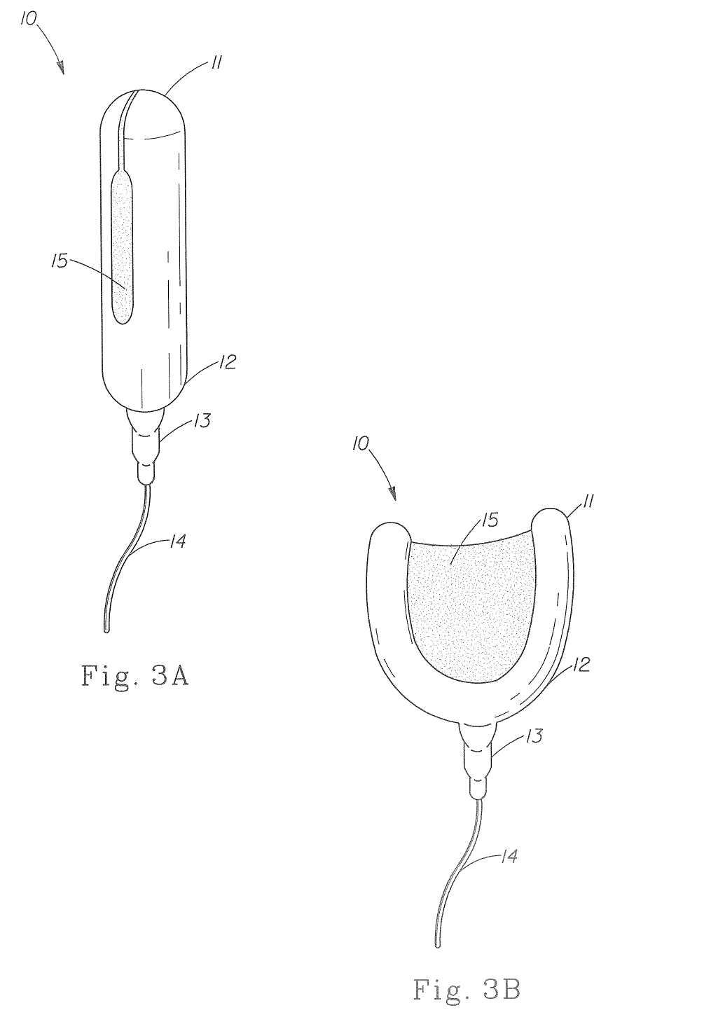

FIG. 3A shows one embodiment of a tampon 10. The tampon 10 has an insertion end 11 and a withdrawal end 12. In this embodiment, the tampon 10 has a secondary absorption member 13. The tampon 10 also has a withdrawal member 14. FIG. 3A shows the tampon 10 in a compressed state. As shown in FIG. 3A, the tampon 10 has a benefit indicator 15. In this embodiment, the benefit indicator 15 is a protection indicator located in a central region of the outer surface of the tampon 10, which can indicate to a user that the tampon 10 can have one or more protection features, such as, e.g., an absorbent core, such as, e.g., an absorbent core that can contain fluid inside the tampon 10. As shown in FIG. 3A, the benefit indicator 15 can continue along the outer surface of the tampon 10 to the insertion end 11 of the tampon 10, which can, for example, indicate to a user that fluid can be drawn from the insertion end 11 of the tampon 10 to the center of the tampon 10, such as, e.g., to the portion of the tampon 10 corresponding to the location of the benefit indicator 15. FIG. 3B shows the tampon 10 of FIG. 3A as expanded, such as, for example, after the tampon 10 is contacted with liquid. As shown in FIG. 3B, the tampon 10 can have a benefit indicator 15 that can communicate a central zone of absorption to a user.

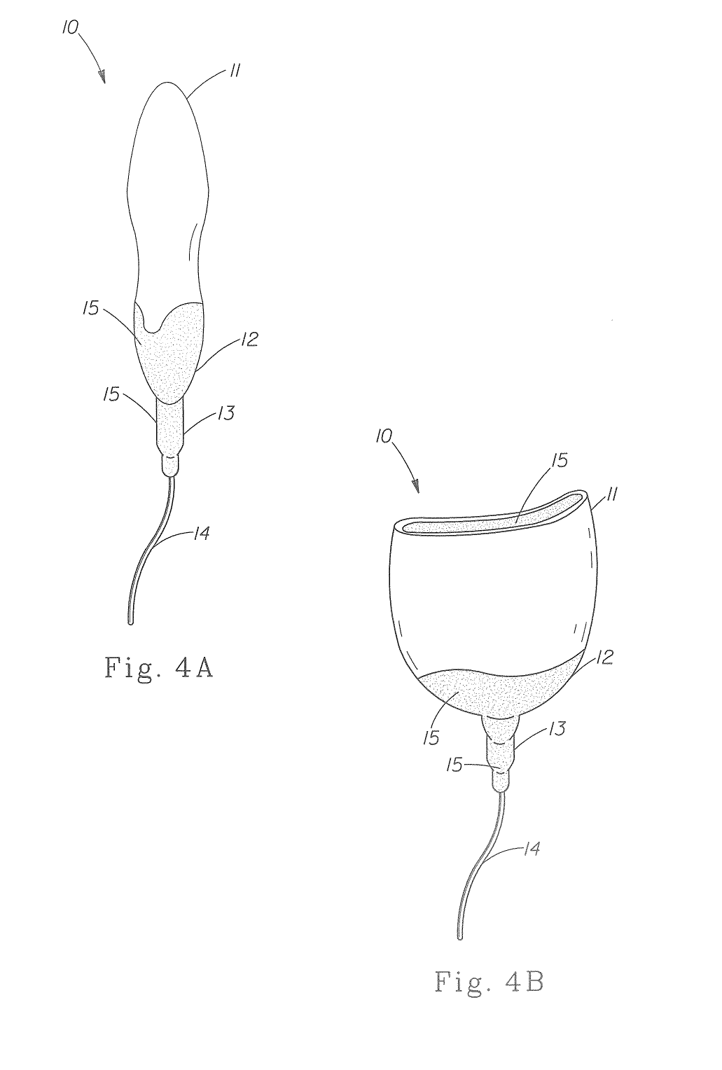

FIG. 4A shows one embodiment of a tampon 10. The tampon 10 has an insertion end 11 and a withdrawal end 12. In this embodiment, the tampon 10 has a secondary absorption member 13. The tampon 10 also has a withdrawal member 14. FIG. 4A shows the tampon 10 in a compressed state. As shown in FIG. 4A, the tampon 10 has a benefit indicator 15. In this embodiment, the benefit indicator 15, such as, e.g., a protection indicator, is located at the withdrawal end 12 of the tampon 10. A benefit indicator 15 located at the withdrawal end 12 of the tampon 10 can, for example, provide a visual barrier line, such as to communicate to a user that fluid preferentially does not travel past the benefit indicator 15. As shown in FIG. 4A, the tampon 10 has a second benefit indicator 15 located on the secondary absorption member 13. In certain embodiments, the second benefit indicator 15 located on the secondary absorption member 13 can communicate an additional visual barrier to a user and/or can emphasize to a user the presence of the secondary absorption member 13. FIG. 4B shows the tampon 10 of FIG. 4A as expanded, such as, for example, after the tampon 10 is contacted with liquid. As shown in FIG. 4B, the benefit indicator 15 can be substantially maintained post-expansion. In addition, the tampon 10 can have one or more additional benefit indicators 15 that can communicate a central zone of fluid containment to a user upon expansion of the tampon 10.

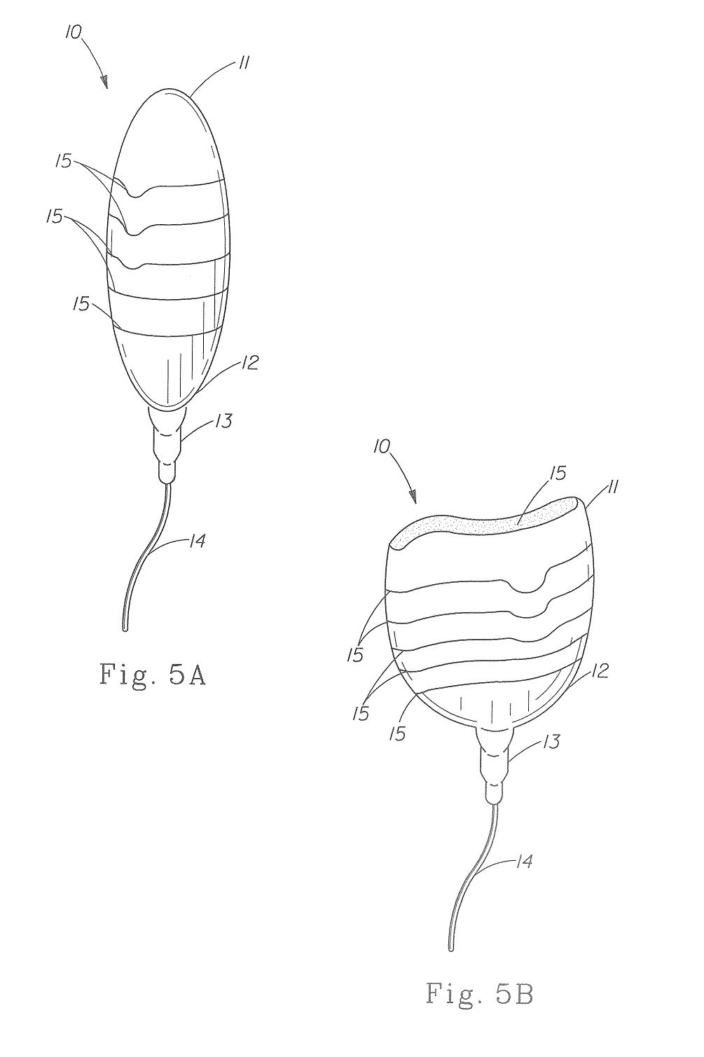

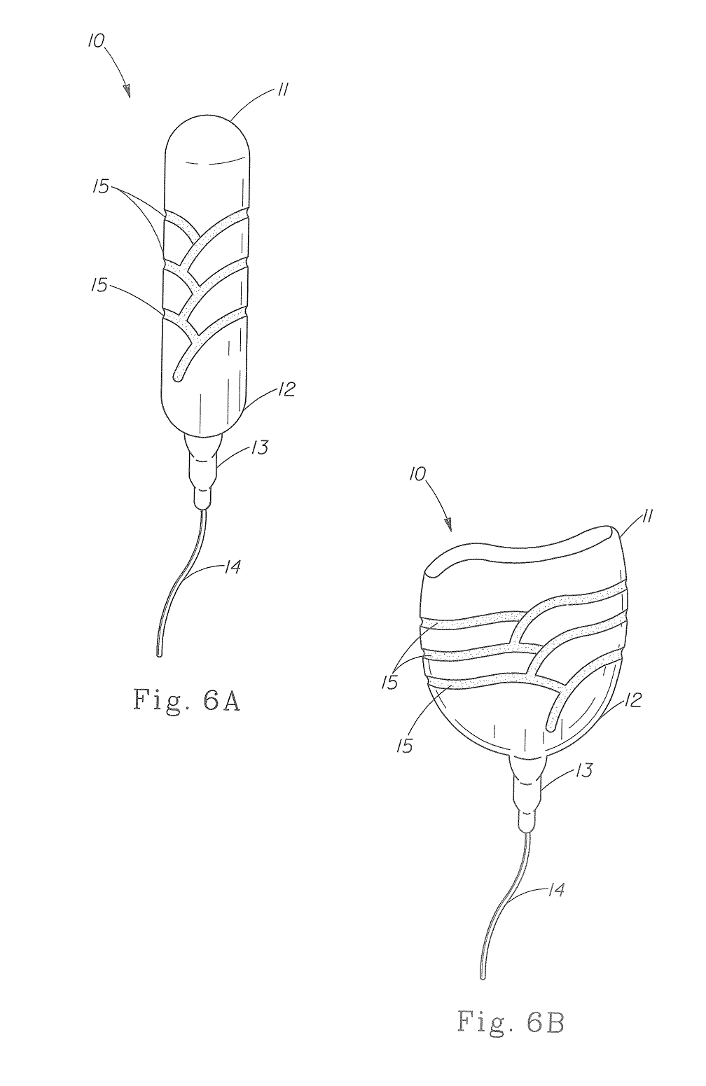

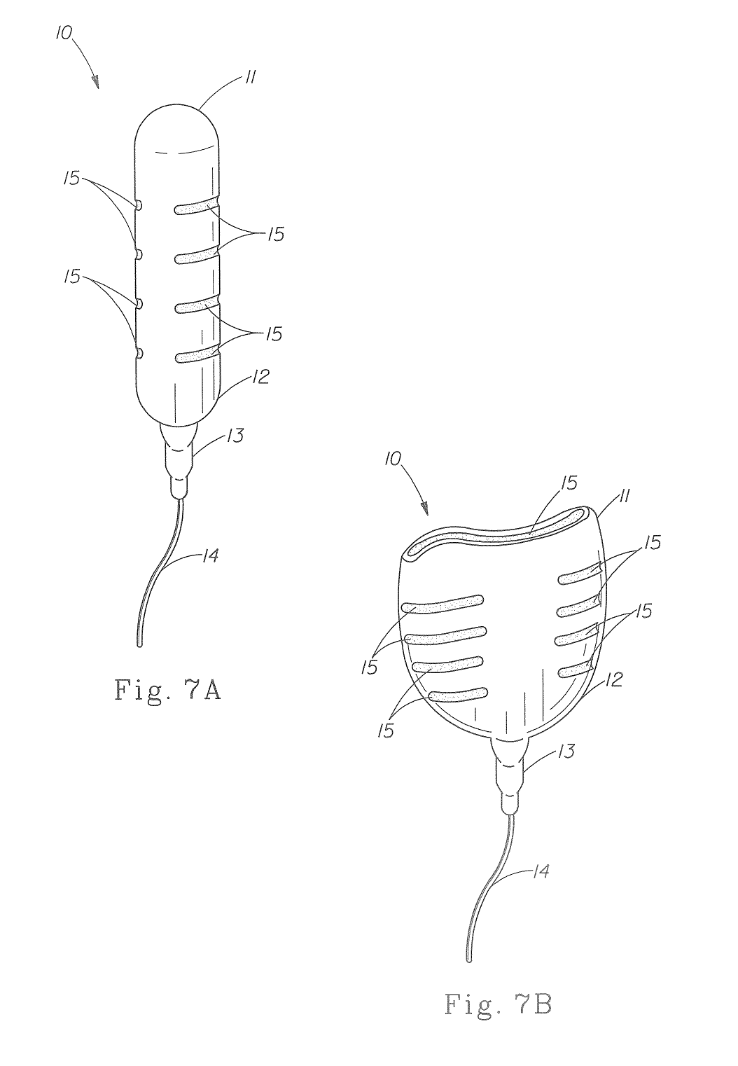

FIGS. 5A, 6A, and 7A show embodiments of tampons 10. The tampon 10 has an insertion end 11 and a withdrawal end 12. The tampon 10 can have a secondary absorption member 13 and/or a withdrawal member 14. As shown in FIGS. 5A, 6A, and 7A, the tampon 10 can have a plurality of benefit indicators 15. In this embodiment, the benefit indicators 15 can correspond to protection features, such as, e.g., channels, protrusions, and/or grooves in the surface of the tampon 10. FIG. 5B shows the tampon 10 of FIG. 5A as expanded, such as, for example, after the tampon 10 is contacted with liquid. FIG. 6B shows the tampon 10 of FIG. 6A as expanded. FIG. 7B shows the tampon 10 of FIG. 7A as expanded. As shown in FIGS. 5B, 6B, and 7B, the benefit indicator 15 can be substantially maintained post-expansion. In certain embodiments, a benefit indicator 15 having a similar shape and/or location pre-expansion and post-expansion can communicate to a user that the benefit, such as, e.g., protection features and/or the perception of protection features, such as, e.g., fluid pathways and/or fluid containment features, can continue during use of the tampon 10.

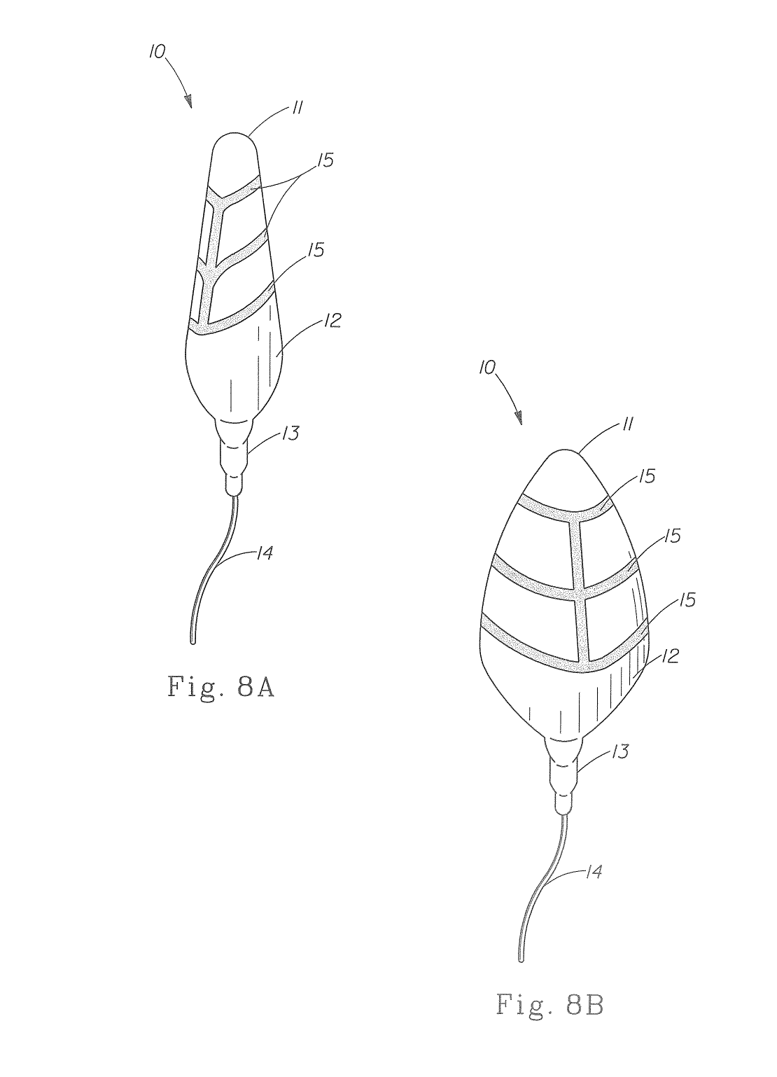

FIG. 8A shows one embodiment of a tampon 10. The tampon 10 has an insertion end 11 and a withdrawal end 12. In this embodiment, the tampon 10 has a secondary absorption member 13. The tampon 10 also has a withdrawal member 14. FIG. 8A shows the tampon 10 in a compressed state. The tampon 10 can have one or more benefit indicators 15. As shown in FIG. 8A, the one or more benefit indicators 15 can communicate to a user the perception of channels and/or fluid pathways, such as, e.g., pathways that can direct fluid to the core of the tampon 10. FIG. 8B shows the tampon 10 of FIG. 8A as expanded, such as, for example, after the tampon 10 is contacted with liquid. As shown in FIG. 8B, the benefit indicator 15 can be substantially maintained post-expansion.

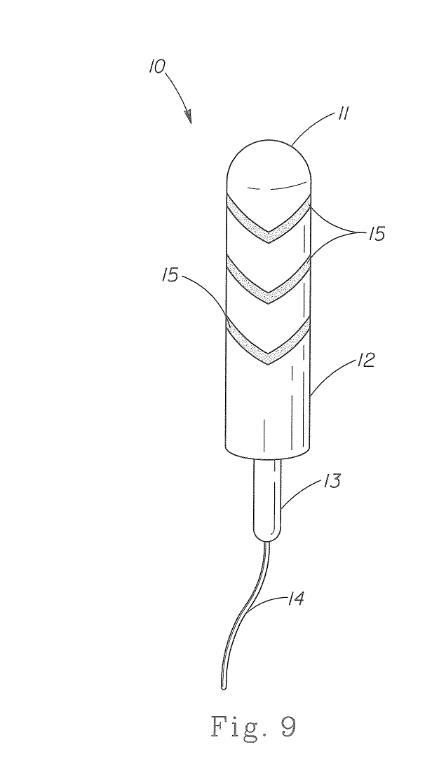

FIG. 9 shows one embodiment of a tampon 10. The tampon 10 has an insertion end 11 and a withdrawal end 12. The tampon 10 can have a secondary absorption member 13 and/or a withdrawal member 14. The tampon 10 can have one or more benefit indicators 15. As shown in FIG. 9, the one or more benefit indicators 15 can correspond to one or more grooves and/or ridges in the tampon 10 and/or the one or more benefit indicators 14 can communicate to a user the perception of a feature, such as, e.g., a protection feature, such as, e.g., the perception of channels, fluid pathways, and/or fluid containment features in the tampon 10.

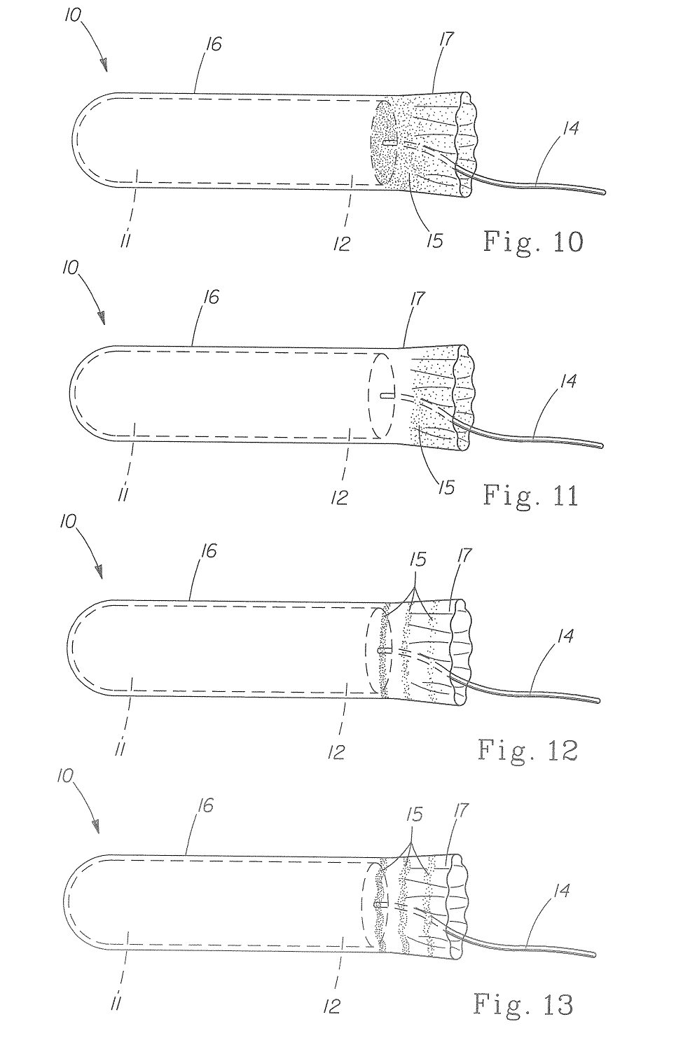

FIG. 10 shows one embodiment of a tampon 10. The tampon 10 has an insertion end 11 and a withdrawal end 12. The tampon also has a withdrawal member 14. As shown in FIG. 10, the tampon 10 can have an overwrap 16 that can extend past the withdrawal end 12 of the tampon 10 to form a skirt 17. The tampon can have one or more benefit indicators 15. As shown in FIG. 10, the one or more benefit indicators 15 can be disposed on the skirt 17. In certain embodiments, the benefit indicator 15 can include a gradient, such as, e.g., a gradient moving from dark to light as the skirt 17 extends away from the withdrawal end 12 of the tampon 10. Such a gradient can, for example, communicate to a user benefits relating to the skirt 17, such as, e.g., additional leakage protection.

FIG. 11 shows one embodiment of a tampon 10. The tampon 10 has an insertion end 11 and a withdrawal end 12. The tampon also has a withdrawal member 14. As shown in FIG. 11, the tampon 10 can have an overwrap 16 that can extend past the withdrawal end 12 of the tampon 10 to form a skirt 17. The tampon can have one or more benefit indicators 15. As shown in FIG. 11, the one or more benefit indicators 15 can be disposed on the skirt 17. In certain embodiments, the benefit indicator 15 can include a colored and/or patterned section at the bottom of the skirt 17. A benefit indicator 15 disposed at the bottom of the skirt 17 can, for example, communicate to a user the perception of a fluid barrier.

FIG. 12 shows one embodiment of a tampon 10. The tampon 10 has an insertion end 11 and a withdrawal end 12. The tampon also has a withdrawal member 14. As shown in FIG. 12, the tampon 10 can have an overwrap 16 that can extend past the withdrawal end 12 of the tampon 10 to form a skirt 17. The tampon can have one or more benefit indicators 15. As shown in FIG. 2 the one or more benefit indicators 15 can be disposed on the skirt 17. In certain embodiments, the one or more benefit indicators 15 can include a series of bands, such as, e.g., a series of colored bands decreasing in intensity of color along the skirt 17. Such indicators can, for example, communicate a perception of fluid containment to a user.

FIG. 13 shows one embodiment of a tampon 10. The tampon 10 has an insertion end 11 and a withdrawal end 12. The tampon also has a withdrawal member 14. As shown in FIG. 13, the tampon 10 can have an overwrap 16 that can extend past the withdrawal end 12 of the tampon 10 to form a skirt 17. The tampon can have one or more benefit indicators 15. As shown in FIG. 13, the one or more benefit indicators 15 can be disposed on the skirt 17. In certain embodiments, the one or more benefit indicators 15 can include a series of indicators, such as, e.g., a series of indicators changing in intensity of color and/or pattern along the skirt 17. Such indicators can, for example, communicate a perception of fluid containment to a user.

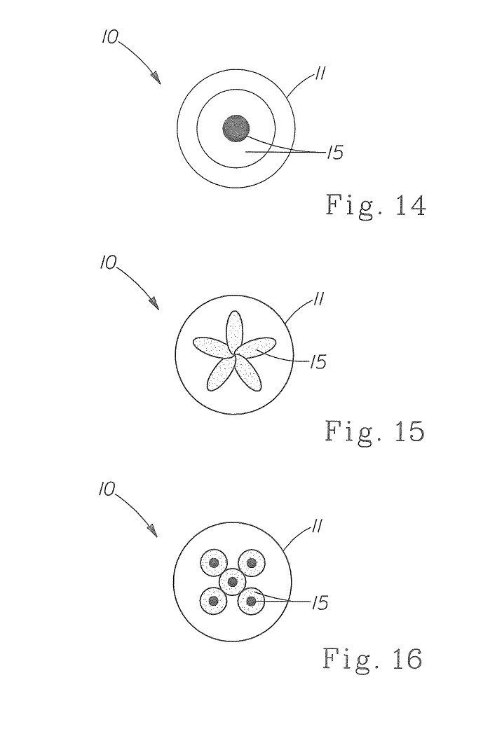

FIGS. 14-16 shows embodiments of insertion ends 11 of tampons 10. The insertion end 11 can have one or more benefit indicators 15. In certain embodiments, the benefit indicators 15 can be aligned with channels and/or can communicate to a user the perception of channels and/or fluid wicking into the core of the tampon 10.

In certain embodiments, the benefit indicator can be a color, such as, e.g., a color in a hue or shade that differs from the portion of the tampon not having an indicator (i.e., the body of the tampon) and/or a contrasting color to the body of the tampon. In certain embodiments, the benefit indicator can be a contrasting color to the body of the tampon, such as, e.g., two different hues. Benefit indicators of a contrasting color can be useful to quickly communicate a tampon feature to a user. In certain embodiments, the difference in color (i.e., ΔE*) between the tampon body and the color of the benefit indicator can be at least about 3.5, at least about 6, at least about 12, at least about 18, at least about 24, at least about 30, or more. The ΔE* can calculated by the formula ΔE*=[(L*_{X.}−L*_{Y})²+(a*_{X.}−a*_{Y})²+(b*_{X.}−b*_{Y})²]^{1/2}, where ‘X’ and ‘Y’ are measured at different points on the viewing surface, such as, e.g., at a point including the indicator and a point not including the indicator. In addition or alternatively, the benefit indicator can be a coordinating color to the tampon body, such as, e.g., two different shades of the same color, such as, e.g., a shade that can be considered pastel and a shade that can be considered bright. A benefit indicator of a coordinating color can be useful to identify a tampon feature to the user while communicating a soothing appearance, an appearance of fun, and/or a seasonal appearance.

A benefit indicator can cover any suitable amount of a surface of the tampon, such as, e.g., the outer surface of the tampon and/or the outer surface of the overwrap. In certain embodiments, a benefit indicator can cover less than substantially the entire outer surface of the tampon, such as, e.g., less than about 90%, less than about 80%, less than about 70%, less than about 60%, less than about 50%, less than about 40%, less than about 30%, less than about 20%, and/or less than about 10% of the outer surface of the tampon. In certain embodiments, a benefit indicator can cover all or a portion of the interior surface of the tampon. Some or all of a benefit indicator disposed on the interior surface of the tampon may not be visible to a user pre-use and/or post-use.

Benefit indicators can be made using any suitable technique. Suitable techniques include, e.g., printing, stamping, coating, impregnating, embossing, folding, incorporated colored fibers into the absorbent article, dimensional coating, three-dimensional coating, flocking, and/or printing heat activated elements. Benefit indicators can include printed indicia, such as printed figures, designs, lines or line segments, flocking, or dimensional coating. In certain embodiments, the benefit indicator can be a non-textual indicator, such as, e.g., one or more colors and/or patterns.

In certain embodiments, the benefit indicator can intuitively communicate to a user one or more features of a tampon, such as, e.g., using non-textual visual indicators. Non-limiting examples of non-textual, intuitive indicators include, e.g., use of patterns, lines, curves, colors, and/or other suitable indicators. While not desiring to be bound to any particular theory, it is thought that the human brain interprets non-textual, intuitive communication based on prior experiences, instinct, emotions and/or feelings that are generated within the human brain when it is exposed to these forms of non-textual, intuitive communication.

Benefit indicators can be any suitable size or shape, such as, e.g., circular, elliptical, square, rectangular, triangular, arced, curved, lines, cross-hatching, stars, teardrops, waves, petal-shaped, or any other conceivable shape able to be readily perceived by a user. The one or more benefit indicators can be formed to have essentially identical size and shape as compared to one or more other benefit indicators on the individual tampon. Alternatively, one or more benefit indicators can have different shapes and/or sizes.

An array of tampons is also provided. The array can include a first tampon having at least a first benefit indicator and a second tampon having at least a second benefit indicator. The tampons can have additional benefit indicators that can be the same or different. For example, in certain embodiments, the first benefit indicator can be a visual indicator such as, e.g., a first color, and the second benefit indicator can be a visual indicator such as, e.g., a second color, wherein the first color and the second color are the same or different. The first benefit indicator can communicate a first feature and the second benefit indicator can communicate a second feature. In certain embodiments, the first benefit indicator can communicate a first feature and the second benefit indicator can also communicate the first feature, such as, e.g., an embodiment where the feature is a channel and/or the perception of a channel and the first benefit indicator is a first color and the second benefit indicator is a second color. The array can be packaged in separate external packages or can be packaged in the same external package. In certain embodiments, the benefit indicators can be one or more protection indicators and the features can be one or more protection features.

Any type of tampon or other article suitable for use with a benefit indicator can be used. Suitable tampons include, e.g., self-sustaining tampons and deformable, fluid-permeable bag tampons. Tampons are generally “self-sustaining” if they will tend to retain their general shape and size before use. A typical self-sustaining tampon can be about 35 to about 60 mm long and about 5 to about 20 mm wide. The deformable, fluid-permeable bag tampon can include pieces such as absorbent chips, spheres, or fibers such that the fluid permeable bag tampon is readily deformable with a force of less than about 3 psi.

Self-sustaining tampons can be formed from absorbent material that can be any suitable shape, size, material, or construction prior to compression and/or shaping. For example, the pledget can include a rolled, tubed, or flat construction of an absorbent that can be a circle, an oval, a semi-circle, a triangle, a chevron shape, an H shape, a bow-tie shape, or any other suitable shape, such as, e.g., shapes described in, for example, U.S. Pat. Nos. 3,738,364; 5,911,712; 6,740,070; 6,887,266; and 6,953,456. The materials for the tampon can be formed into a fabric, web, or batt that can be suitable for use in the absorbent material by any suitable process such as airlaying, carding, wetlaying, hydroentangling, needling or other known techniques.

The absorbent material can be a laminar structure comprised of integral or discrete layers. In other embodiments, the pad need not have a layered structure at all. The absorbent material can comprise a folded structure, a rolled structure, or any other suitable structure. The resulting compressed absorbent member of the tampon can be constructed from a wide variety of liquid-absorbing materials commonly used in absorbent articles. Such materials include, for example, rayon (such as GALAXY rayon (a tri-lobed rayon) or DANUFIL rayon (a round rayon), both available from Kelheim Fibres GmbH of Kelheim, Germany), cotton, folded tissues, woven materials, nonwoven webs, synthetic and/or natural fibers or sheeting, comminuted wood pulp, which is generally referred to as airfelt, foams, or combinations of these materials. Examples of other suitable materials include: creped cellulose wadding; meltblown polymers including coform; chemically stiffened, modified or cross-linked cellulosic fibers; synthetic fibers such as crimped polyester fibers; peat moss; foam; tissue including tissue wraps and tissue laminates; or any equivalent material or combinations of materials, or mixtures of these. Additionally, superabsorbent materials, such as superabsorbent polymers or absorbent gelling materials can be incorporated into the tampon.

In certain embodiments, all or a portion of the tampon can be compressed into a substantially cylindrical configuration, however, other shapes can be used. Suitable tampons can have any suitable cross-sectional shape, such as, e.g., circular, generally circular, circular with flattened sides, elliptical, oval, or any other suitable cross-sectional shape. In addition, or alternatively, the tampon can have varying cross-sectional shapes and/or sizes. Exemplary tampons having a circular cross-section are described in, e.g., U.S. Pat. Nos. 6,837,882; 6,740,070; 6,682,513; 6,599,279; 6,554,814; and 6,258,075. Tampons have an oval cross-sectional shape are described in, e.g., U.S. patent application Ser. No. 11/703,946, filed Feb. 8, 2007. In certain embodiments, the tampon can be a shaped tampon, such as, e.g., disclosed in U.S. Pat. No. 6,824,536.

The tampon can include one or more withdrawal members. The withdrawal member can be joined to the tampon and/or can be graspable by the user for removal after use. Any suitable withdrawal member can be used. Suitable withdrawal members include, e.g., string, cord, ribbon, loop, tab, or the like. The tampon can also or alternatively include one or more secondary absorbent members, such as, for example, a mass of secondary absorbent material attached to the withdrawal cord proximate the withdrawal end of the tampon. Suitable secondary absorbent members are described in, e.g., U.S. Pat. No. 6,258,075.

The tampon can alternatively, or in addition, include a fluid pervious overwrap. The overwrap can include any suitable material, such as, e.g., rayon, cotton, bicomponent fibers, polyethylene, polypropylene, other suitable natural or synthetic fibers, and mixtures thereof. In certain embodiments, all or a portion of the overwrap can be treated to be hydrophilic, hydrophobic, wicking or non-wicking. In certain embodiments, the overwrap can substantially cover the absorbent material. In addition, or alternatively, the overwrap can extend beyond the withdrawal end of the tampon to form a skirt. The skirt can extend any suitable distance beyond the withdrawal end of the absorbent material, such as, e.g., from about 2 mm to about 30 mm, from about 5 mm to about 20 mm, or any other suitable length. In certain embodiments, the skirt need not be an extension of the overwrap and can be attached to the withdrawal end of the absorbent material of the tampon in any suitable manner.

The tampon can be inserted in any suitable manner, such as, e.g., digitally or using an applicator. Any type of applicator suitable for insertion of a feminine hygiene article can be used, such as, e.g., telescoping, insertion tube and plunger, and compact applicators. In certain embodiments, the applicator can be a self-orienting applicator, such as, e.g., described in U.S. patent application Ser. No. 11/703,919, filed Feb. 8, 2007. Suitable applicator materials include, for example, paper, paperboard, cardboard, cellulose, such as, e.g., molded cellulose, or any combinations thereof, polyethylene, polypropylene, polybutylene, polystyrene, polyvinylchloride, polyacrylate, polymethacrylate, polyacrylonitrile, polyacrylamide, polyamide, nylon, polyimide, polyester, polycarbonate, polylactic acid, poly hydroxyalkanoate, ethylene vinyl acetate, polyurethane, silicone, derivatives thereof, copolymers thereof, mixtures thereof, or any suitable smooth plastic material. Examples of suitable materials are disclosed in, e.g., U.S. Pat. Nos. 5,346,468 and 5,558,631. In certain embodiments, additives can be included in the material to alter or enhance certain material properties. Suitable additives include, for example, mold release agents, slip agents, surface energy modifiers, pearlescent agents, and/or any other suitable additives. In certain embodiments, the insertion member can be coated with a substance to give it a high slip characteristic, such as, e.g., with wax, polyethylene, a combination of wax and polyethylene, cellophane, clay, mica, and other lubricants that can facilitate comfortable insertion. Alternatively, or in addition, the insertion member can include a textured surface. Texture can be provided in any suitable manner, such as, e.g., by designing texture into or adding texture to the insertion member.

In certain embodiments, the applicator can be configured to enable the user to observe at least a portion of or substantially the entire benefit indicator. One technique that allows the user to better notice the benefit indicator is to make visual observation possible through the use of translucent or even transparent applicator materials. In certain embodiments, substantially the entire applicator can be translucent and/or transparent. Alternatively, or in addition, the applicator can include a translucent and/or transparent insertion end, such as, e.g., a cap or covering made of film or other suitable material. Exemplary applicators are described in, e.g., U.S. Pat. No. 6,932,805.

In certain embodiments, the applicator can include one or more portions, such as, e.g., the gripping portion, the insertion member, and/or the plunger, having one or more indicia, such as, e.g., one or more colors, disposed on the applicator. The one or more indicia can be coordinated with and/or can substantial match one or more of the benefit indicators on the tampon. Any suitable amount of the gripping portion, insertion member, and/or plunger can include one or more indicia, such as, e.g., one or more colors. Suitable amounts include, e.g., less than about 90%, less than about 80%, less than about 70%, less than about 60%, less than about 50%, less than about 40%, less than about 30%, less than about 20%, less than about 10%, or less.

The tampon can be packaged in a wrapper, such as, e.g., individually packaged in a wrapper. The tampon and/or applicator may or may not be visible through the wrapper. In certain embodiments, the applicator can be individually packaged in a wrapper and a plurality of wrapped applicators can be packaged in a container such as, e.g., a bag, a box, and/or a carton. The wrapped tampons may or may not be visible to the user through a window in the package.

The package can be any suitable shape, such as, for example, a square, a rectangle, a polyhedral shape, and/or a circular shape. In certain embodiments, the package can comprise an interior surface and an exterior surface. The interior surface can define an interior space. The exterior surface of the package can have at least a front and a back, wherein the front of the package can be the side typically viewed first by a user, such as, e.g., the side predetermined to be viewed first by a user.

The package can be made of any suitable material. In certain embodiments, the package can comprise one or more inflexible materials, such as, for example, cardboard, paperboard, cartonboard, chipboard, plywood, SBS, metal, plastic, paper, card stock, fabric, ceramic, polymer, natural or synthetic fibers, webs, mesh, screen, wood, composite, mixtures or combinations thereof, or any other suitable material. Alternatively, or in addition, the package can be made of a flexible material, such as, e.g., blown or cast film in a blend of low density polyethylene and linear low density polyethylene, metallocenes, ethylene vinyl acetate, surlyn, polyethylene terephthalate, biaxally oriented polypropylene, nylon, combinations thereof, or any other suitable material.

The package can include at least one window, such as, e.g., a region of transparency. As used herein, “transparency” means having the property of transmitting light without substantial scattering so that items lying beyond can be visible to a person. The window can comprise a transparent covering, such as, for example, a transparent film. In addition, or alternatively, the window can comprise an opening in the package that does not include a covering. In certain embodiments, the window can comprise a transparent covering that can be clear, translucent, tinted and/or opaque. The transparent covering can be tinted in any suitable color or combination of colors. The transparent covering can also, or alternatively, be printed and/or embossed. In certain embodiments, the window can be one or more images that depict and/or give the perception of a window, such as, e.g., one or more images that depict a region of transparency and/or an opening.

The window can be disposed on at least one side, such as, e.g., the front, the back, and/or one or more sides. In certain embodiments, the window can be disposed on more than one side. The window can be at any location on the side and can comprise any appropriate amount of the package side, such as, for example, from about 5% to about 95% of the side, such as, for example, about 5% to about 75%, about 5% to about 50%, about 5% to about 25%, or any other suitable amount of the side. In certain embodiments, the window can be located in the central region of a side, such as, for example, wherein the window is not located at the edge of the side. In certain embodiments, one or more tampons can be positioned adjacent the window such that at least a portion of one or more of the tampons can be visible through the window.

The tampon, applicator, wrapper, and/or external package can be coordinated. For example, tampons having one or more benefit indicators can be disposed in applicators having a visual signal or indicia that corresponds in visual distinction to the benefit indicator of the tampon and can be packaged in wrappers and/or boxes or cartons bearing a visual signal or indicia that corresponds in visual distinction to the benefit indicator of the tampon. Thus, if the benefit indicator is a shade of color, visual indicia on the wrapper and/or package can be a matching or substantially-matching shade of color. By “substantially-matching” is meant the color is close enough that the benefit indicator and the wrapper and/or packaging can be easily matched by one comparing tampons and packaging. For example, substantially-matching shades can be matching within the range of normal variance of colors from lot to lot of ink, dye, or other color-inducing medium, or within normal variance due to slight differences perceived on film versus paper, and the like. Other means of obtaining corresponding visual distinction include matching the shapes, styles, or overall appearance of visual indicia with corresponding benefit indicators. Thus, in certain embodiments, a user of feminine hygiene articles can choose a tampon having a desired feature more easily based on the wrapper and/or packaging, with a confirmation or reinforcement of that feature on each tampon inside the wrapper and/or packaging. In certain embodiments, the tampon can be co-packaged and/or co-marketed with one or more feminine hygiene articles, such as, e.g., a liner, a sanitary napkin, an interlabial pad, a wipe, or other suitable article. In addition, the tampon can be co-packaged and/or co-marketed with one or more feminine hygiene articles having one or more indicia that can coordinate and/or substantially match one or more benefit indicators on the tampon.

An array of packages is also provided. Each package in the array can include a tampon having one or more benefit indicators optionally disposed within an applicator, the tampon and/or applicator being disposed within a wrapper. The wrapper can optionally be disposed within an external package. In certain embodiments, the benefit indicator, applicator, wrapper, and/or package can be matching and/or coordinating, such as, e.g., having a matching, substantially matching, and/or coordinating shade of a color and/or pattern on at least a portion of the item. Each package in the array can include a coordinating and/or matching tampon, applicator, wrapper, and/or package. In certain embodiments, the first package can have a tampon, applicator, wrapper, and/or package having a first color, the second package can have a tampon, applicator, wrapper, and/or package having a second color, the third package can have a tampon, applicator, wrapper, and/or package having a third color, and so on.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A tampon comprising a tampon body having an insertion end, a withdrawal end, and an outer surface;
the tampon further comprising one or more visually perceptible benefit indicators that are visible pre-use, the one or more visually perceptible benefit indicators being directly disposed on a portion of the outer surface of the tampon, the one or more benefit indicators being a color in a hue or shade that differs from a white color of a portion of the outer surface of the tampon not having an indicator,
wherein the one or more benefit indicators communicate one or more benefits of the tampon to a user, wherein the one or more benefits are layers, a secondary absorbent, channels, grooves, protrusions, a skirt, apertures, an absorbent core, and/or the perception of a fluid barrier, fluid flow path, wicking, and/or fluid containment.

2. The tampon of claim 1, the tampon having an overwrap, wherein the one or more benefit indicators are disposed on the overwrap.

3. The tampon of claim 1, the tampon having a skirt, wherein the one or more benefit indicators are disposed on the skirt.

4. The tampon of claim 1, wherein the one or more benefit indicators are disposed on the insertion end.

5. The tampon of claim 1, the tampon having a secondary absorbent, wherein the one or more benefit indicators are disposed on the secondary absorbent.

6. The tampon of claim 1, wherein the one or more benefit indicators are disposed over less than about 50% of the outer surface.

7. A tampon comprising a tampon body having an insertion end, a withdrawal end, and an outer surface,
the tampon further comprising a first visually perceptible benefit indicator that is visible pre-use; and
a second visually perceptible benefit indicator that is at least partially visible post-use,
wherein the first benefit indicator and the second benefit indicator communicate one or more benefits of the tampon to a user.

8. The tampon of claim 7, wherein the first benefit indicator is at least partially visible post-use.

9. The tampon of claim 7, wherein the second benefit indicator is not visible pre-use.

10. The tampon of claim 7, wherein the first benefit indicator and the second benefit indicator are one or more colors.

11. A tampon comprising a tampon body having an insertion end, a withdrawal end, and an outer surface;
the tampon further comprising a first visually perceptible protection indicator that is visible pre-use, the first visually perceptible protection indicator being directly disposed on a portion of the outer surface of the tampon body substantially between the insertion end and the withdrawal end and substantially symmetrically about a longitudinal and/or transverse axis of the tampon, the first protection indicator being a shade of a color that differs from a white color of a portion of the outer surface of the tampon not having an indicator,
wherein the first protection indicator communicates one or more protection features of the tampon to a user, wherein the one or more protection features are layers, a secondary absorbent, channels, grooves, protrusions, a skirt, apertures, an absorbent core, the perception of a fluid barrier, the perception of fluid flow path, the perception of wicking, and/or the perception of fluid containment.

12. The tampon of claim 11, the tampon further comprising a second benefit indicator that is substantially the same as the first benefit indicator.

13. The tampon of claim 12, wherein the second benefit indicator is disposed on an opposite side of the tampon from the first benefit indicator.

14. The tampon of claim 12, wherein the second benefit indicator is disposed on a same side or sides of the tampon as the first benefit indicator.
